(19) United States
(12) Patent Application Publication
(10) Pub. No.: US 2021/0174969 A1
(43) Pub. Date: Jun. 10, 2021

(54) CELLULAR AUTOMATA MODEL OF STEM-CELL-DRIVEN GROWTH OF SPINAL CORD TISSUE

(71) Applicant: Northeastern University, Boston, MA (US)

(72) Inventors: Gunther Zupanc, Auburndale, MA (US); David Lehotzky, Boston, MA (US); Rifat Sipahi, Brookline, MA (US)

(21) Appl. No.: 17/085,210

(22) Filed: Oct. 30, 2020

Related U.S. Application Data
(60) Provisional application No. 62/928,751, filed on Oct. 31, 2019.

(51) Int. Cl. G16H 50/50

(52) U.S. Cl. CPC G16H 50/50

(57) ABSTRACT

An algorithm is disclosed for computational modeling of stem-cell-driven tissue growth of adult spinal cord tissue. Simulations based on this model can be used for parameter testing and making predictions about the growth dynamics of biological spinal cord tissue. Such predictions include alterations in the growth dynamics and the final properties of the tissue induced by experimental or medical intervention.

### CROSS REFERENCE TO RELATED APPLICATION

This application claims priority from U.S. Provisional Patent Application No. 62/928,751 filed on Oct. 31, 2019 entitled Cellular Automata Model of Stem-Cell-Driven Growth of Spinal Cord Tissue, which is hereby incorporated by reference.

### GOVERNMENT SUPPORT

This invention was made with government support under Grant No. 1538505 awarded by the National Science Foundation. The government has certain rights in the invention.

### BACKGROUND

Modeling approaches have not been employed in biomedical research related to spinal cord injury. However, in other areas of biomedicine, mathematical and computational modeling has become a standard approach over the last few decades.

### SUMMARY

Various embodiments disclosed herein relate to an algorithm for computational modeling of stem-cell-driven tissue growth of adult spinal cord tissue. Simulations based on this model can be used for parameter testing and making predictions about the growth dynamics of biological spinal cord tissue. Such predictions include alterations in the growth dynamics and the final properties of the tissue induced by experimental or medical intervention. The computational modeling has significant potential for making the development of biomedical therapies for the treatment of spinal cord injury more rational and efficient, thereby streamlining this process.

In accordance with one or more embodiments, a computer-implemented method is disclosed for predicting stem-cell-driven growth of biological spinal cord tissue under given conditions. The method includes the steps of: (a) electronically accessing a computational model from a computer storage for simulating stem-cell driven spinal cord tissue growth, the model based on a cellular automata (CA) framework comprising a plurality of lattice sites, wherein each lattice site is empty or contains a cell having one of four state values: stem cell, progenitor cell, differentiated cell, or dead cell; the model including a plurality of interaction rules for predicting the state value of each lattice site at a next time iteration based on the state values of cells in neighboring lattice sites at a current time iteration; (b) 65238398.1 electronically receiving input data specifying an initial lattice composition and lattice boundary conditions; (c) electronically running a simulation on the computational model based on the input data for a plurality of time iterations to predict development of the biological spinal cord tissue over the time iterations; and (d) electronically outputting data on the development of the biological spinal cord tissue.

### BRIEF DESCRIPTION OF THE DRAWINGS

FIGS. 1A, 1B, and 1C illustrate the transformation of the three-dimensional grid into a two-dimensional grid for the cellular automata model in accordance with one or more embodiments.

FIG. 2 is a flowchart illustrating an exemplary algorithm according to which the cellular automata model operates.

FIGS. 3A and 3B are graphs showing the probability of mitosis P_{M}(p) (a) and of the probability of death of daughter cell P_{D}(p) (b) on population pressure p.

FIGS. 4A and 4B are graphs showing the dependence of the probability of activation P_{A}(y) (FIG. 4A) and of the probability of symmetric division P_{S}(y) (FIG. 4B) of stem cells on radial distance y from the central canal.

FIGS. 5A, 5B, and 5C show visualization of extended neighborhood around the middle lattice site.

FIG. 6 is a diagram showing the process of daughter cell placement.

FIGS. 7A and 7B show the process of shifting and placement of daughter cells.

FIG. 8 shows the result of an exemplary simulation at time iteration t=2466, using the parameter values, initial conditions and boundary conditions disclosed herein.

FIG. 9 is a table (Table 1) showing biological properties and rules implemented in the cellular automata model for cell types that are used to study spinal cord growth.

FIG. 10 is a block diagram illustrating an exemplary computer system in accordance with one or more embodiments in which the processes described herein may be implemented.

### DETAILED DESCRIPTION

Growth of spinal cord tissue is a complex biological phenomenon. To gain a deeper understanding of the theory of this phenomenon and to be able to make predictions about the outcome of experimental and biomedical interventions, we have developed a computational model that is able to simulate, with high precision, the outcome of the underlying biological processes. This tool has significant potential to make the development of therapeutic strategies (including the design of drugs) aimed at curing spinal cord injury more rational and cost effective.

The processes disclosed herein complement existing approaches based on in vivo or in vitro testing of the effect of experimental or pharmacological interventions to improve spinal cord regrowth after injury. Due to the predictability power of our invention, the design of such tests can be made more rational, and thus more cost effective. The modeling and simulation software even allows one to make predictions about the likely outcome of experiments that are difficult to perform due to resource constraints. Modifications of this technology can be applied to drug discovery related to tumor growth.

In the following, we describe the underlying mathematical model and outline the algorithm, which has been implemented in form of a MATLAB script. We also demonstrate the feasibility of this modeling approach by showing the results of a simulation based on the MATLAB script.

### Cellular Automata Model

Our model employs the cellular automata (CA) framework in which time, space, and state are treated as discrete variables. In this mathematical framework, a finite number of agents equipped with states interact with each other on a uniform lattice. Each agent occupies one lattice space and its state at the current time iteration is determined by interaction rules dependent on state values of agents within its neighborhood prior to the current time iteration. During each time iteration, the state of an agent can take one value out of a finite set. Therefore, not only time and space but also the states of agents are represented by discrete values.

Using the CA framework, we developed a model for stem-cell-driven tissue growth of the spinal cord. Using this model, computer simulations can be run for predictions about development of biological tissues under different conditions. Below, we describe the steps of model construction in detail.

### Grid

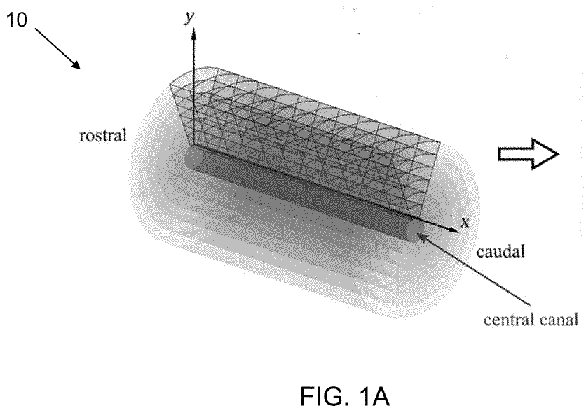

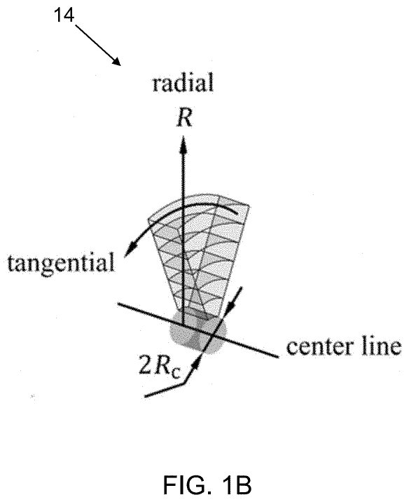

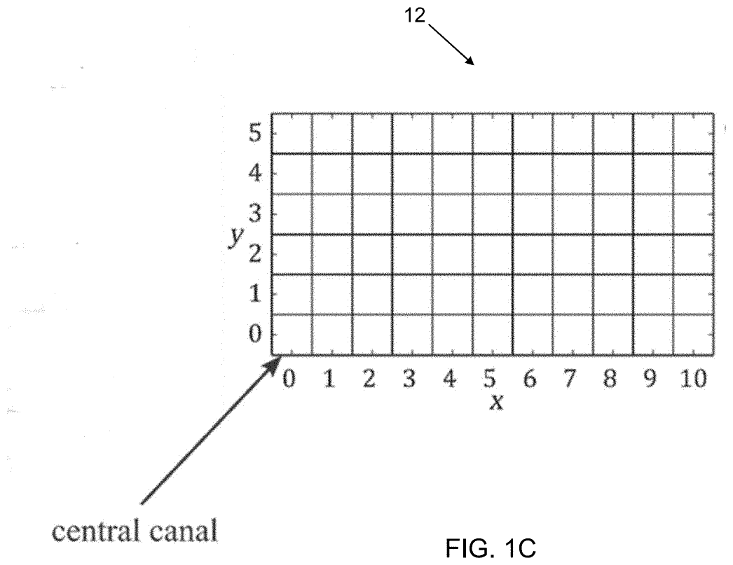

Our CA model is formulated on a two-dimensional (2D) lattice comprised of square lattice sites. Each lattice site is either empty or associated with an agent, which will be referred to as “cell.” This 2D model (FIG. 1C) is related to a three-dimensional (3D) cylindrical model (FIG. 1A), using the following assumptions.
a) There is angular symmetry in cellular composition around the central canal (with symmetry axis being the center line of the central canal).
b) The volume-per-cell ratio shrinks in a hyperbolic manner as the central canal is radially approached, i.e., the volume of lattice sites is inversely proportional to the radial distance from the center line of the central canal.

These two assumptions allow the transformation of the 3D problem into a 2D one. Note that the lattice structure assumes a specific regular, axisymmetric topology to be present inside the 3D tissue. The radial size of 3D lattice sites is fixed, and equal to the size of lattice sites in axial direction x. Hence, we have square lattice sites in the 2D grid (FIG. 1C). By contrast, the tangential size of 3D lattice sites increases with radial distance R=R_{c}+y+½ measured from the center line of central canal (FIG. 1B), where y is the radial distance of center points of lattice sites measured from the central canal surface and R_{c} is the radius of central canal.

FIGS. 1A, 1B, and 1C illustrate the transformation of the three-dimensional grid 10 (FIG. 1A) into a two-dimensional grid 12 (FIG. 1C). The rostro-caudal axis x, and radial axis y measured from the central canal are divided into equidistant segments. The resulting two-dimensional square lattice corresponds to one angular slice of the three-dimensional grid. In each rostro-caudal segment 14 (FIG. 1B) of the three-dimensional grid, the tangential size of lattice sites elongates with the increase of radial distance R from the center line of central canal, whose radius is denoted by Rc.

### Rules

Rules describing the cellular mechanisms of cell activation, division, differentiation, apoptosis, and phagocytosis are adopted from the simple neurosphere model (Sipahi and Zupanc 2018), with the following significant improvements:
a) Previous mathematical modeling of spinal cord growth has implicated population pressure as a critical parameter (Ilie [symbol] et al. 2018). Thus, in the current CA model a generalized notion for population pressure p is developed and quantified using the cellular density of the extended neighborhood of lattice sites, i.e., by taking into account cellular density beyond the four immediate neighbors, also known as von Neumann neighbors.
b) Probability of death of daughter cell P_{D}(p) and probability of mitosis P_{M}(p) functions are introduced to characterize apoptosis and mitosis of cells, respectively.
c) The probability of stem cell activation P_{A}(y) is dependent on radial distance y from the central canal surface.
d) Stem cells can undergo symmetric division with probability P_{S}(y), which depends on radial distance y from the central canal surface.

For consistency of notation, throughout this description we denote all probabilities with uppercase letter P and all population pressure values with lowercase letter p.

### States

Each lattice site is either empty or contains a cell whose state can take one of the following values:
a) Stem cell: either activated or quiescent. At iteration number t∈ [symbol] (t is a natural number), integer numbers qₜ>0 are assigned to stem cells counting the number of iterations elapsed since their last division.
b) Progenitor cell: at iteration number t∈ [symbol] , integer numbers hₜ>0 are assigned to progenitor cells counting the number of divisions they have undergone since their birth.
c) Differentiated cell: no time-dependent quantity is assigned to differentiated cells as they cannot divide and cannot die.
d) Dead cell: at iteration number t∈ [symbol] , integer numbers dₜ>0 are assigned to dead cells counting the number of iterations elapsed since their death occurred.

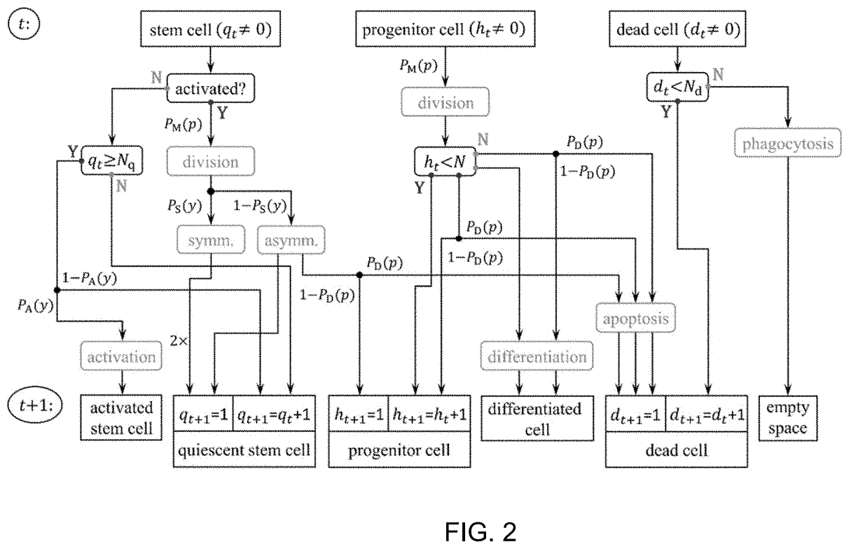

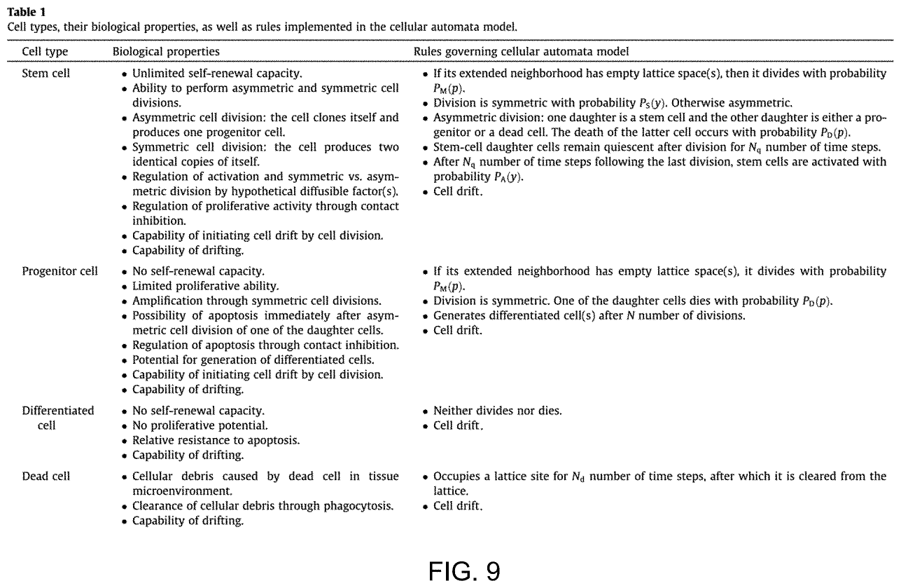

The rules describing how these state values are updated in one iteration step, i.e., between iteration numbers t and t+1, are summarized in flowchart (FIG. 2) and tabular (Table 1—FIG. 9) formats.

FIG. 2 illustrates the algorithm according to which the cellular automata model operates. States whose values can be updated in the next iteration step and correspond to iteration number t, are indicated at the top. All possible updated state values at iteration number “t+1” are given at the bottom of the figure. Outcomes (yes/no) of logical operations are indicated with Y and N, respectively. At junctions marked by dots, choices between two paths are made randomly with the indicated probabilities. Associated biological events are indicated.

Table 1 shows the biological properties and rules implemented in the cellular automata model for cell types that are used to study spinal cord growth. For each cell type only those properties are listed that are relevant for model implementation.

### Probabilities

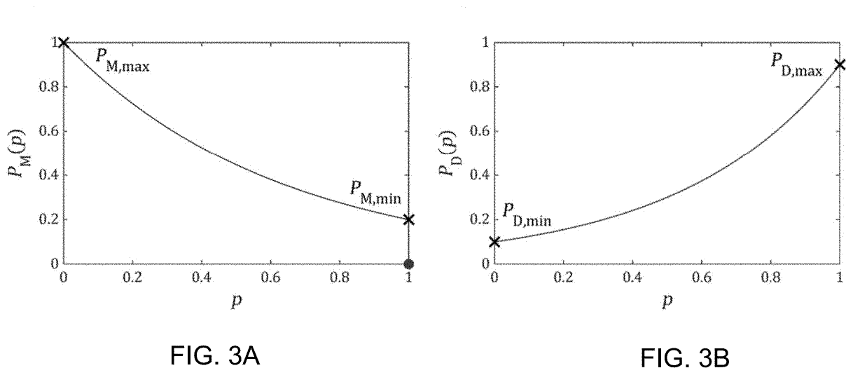

As mentioned above, the probabilities mediating cell activation, division and apoptosis are dependent on either population pressure p or radial distance y from the central canal. In our model the former dependence is characterized by two-parameter while the latter by single-parameter functions. In particular, probabilities related to population pressure are characterized by either linear, or exponential functions (for specific formulae see Appendix 1). These P_{χ}(p), χ=M (Mitosis), and D (Death) functions are parameterized using probabilities P_{χ,min} and P_{χ,max} at minimum (p=0) and maximum (p=1⁻) population pressure values, corresponding to cases when the cell's extended neighborhood is completely empty or completely filled, respectively. Furthermore, the probability of mitosis function P_{M}(p) is characterized such that mitosis is not possible when extended neighborhood of the cell is completely filled. Consequently, P_{M}(1⁺)=0, always holds which can lead to discontinuity in function P_{M}(p). Contrariwise, function P_{D}(p) is always continuous, thus P_{D}(1⁺)=P_{D}(1⁻) always holds. For a particular parameter setting, P_{χ}(p), χ=M, D functions are plotted in FIGS. 3A and 3B.

FIGS. 3A and 3B show the probability of mitosis P_{M}(p) (FIG. 3A) and of the probability of death of daughter cell P_{D}(p) (FIG. 3B) on population pressure p, with the assumption of exponential characteristics and using parameter values P_{M,min}=0.2, P_{M,max}=¹, and P_{D,min}=0.1, P_{D,max}=0.9, respectively. These parameter values are indicated by “x” marks.

Functions describing the probability of activation P_{A}(y) and symmetric division P_{S}(y) of stem cells are dependent on radial distance y measured from the central canal. In both cases hyperbolic dependence on y is assumed, which results from the following assumptions.
a) Both activation and symmetric division are mediated by a diffusive factor evenly distributed inside the volume of the spinal cord.
b) The overall amount of diffusive factor inside a particular 3D lattice site occupied by a stem cell is inversely proportional to the probability of activation and symmetric division of the stem cell.

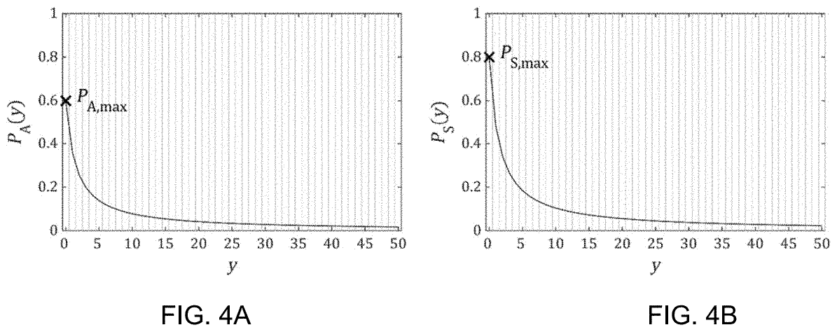

Due to the axisymmetric nature of the model, the volume of lattice sites increases proportionally with respect to radial distance R=R_{c}+y+½ measured from the center line of central canal. As a result, the final formula for the probability of activation and symmetric division of stem cells is of the form

$${P}_{χ} ( y ) = \frac{{P}_{χ , max}}{1 + 2 y \text{/} ( 2 {R}_{c} + 1 )} , \qquad ( 1 )$$

where χ=A (Activation), S (Symmetric Division) (for more details on derivation see Appendix 2). The radius of central canal R_{c} can be provided by experimental data, therefore these functions are characterized by a single parameter P_{χ,max} which is the maximum probability corresponding to y=0, i.e., to lattice sites located along the surface of the central canal. In FIGS. 4A and 4B, these probability functions are visualized for a particular parameter setting.

FIGS. 4A and 4B show the dependence of the probability of activation P_{A}(y) (FIG. 4A) and of the probability of symmetric division P_{S}(y) (FIG. 4B) of stem cells on radial distance y from the central canal, using parameter values P_{A,max}=0.6, and P_{S,max}=0.8, respectively. These parameter values are indicated by “x” marks. The radius of central canal was set to R_{c}=1.

### Population Pressure

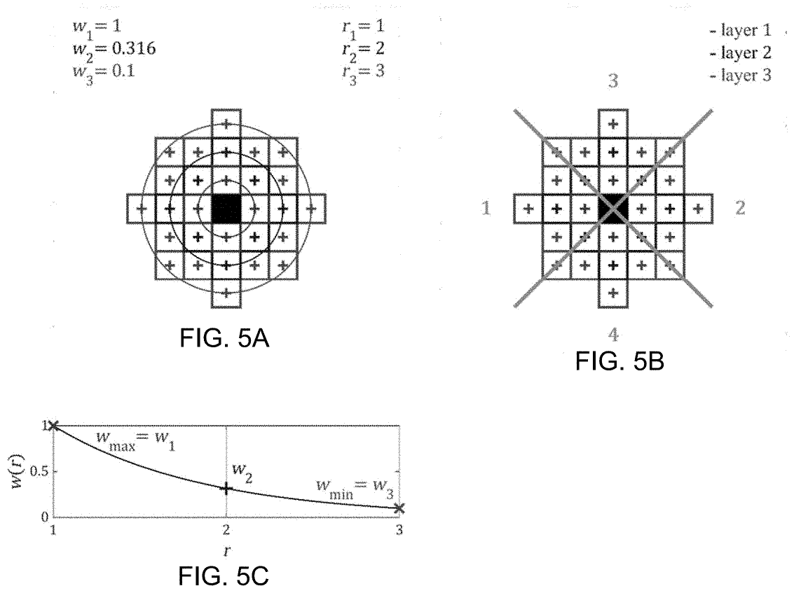

As described above, the probability of mitosis and death of daughter cell are mediated by population pressure p. In our model, population pressure is a quantity that is assigned to the center point of each lattice site. It characterizes the occupancy of the extended neighborhood of an associated lattice site taking into account its distance from cells located inside the extended neighborhood. In particular, neighborhood layers (see FIG. 5A) are defined by radii rₖ=k, k=0, 1, . . . , Nₗ; with Nₗ being the number of layers. In the extended neighborhood, lattice sites whose center point locations satisfy r∈]rₖ₋₁, rₖ] belong to layer k, k=1, . . . , Nₗ; where r is the distance measured between the center point of a lattice site in the extended neighborhood and the center point of the lattice site to which population pressure p is assigned. The occupancy rate of each neighborhood layer is weighed using weights wₖ=w(rₖ) determined by a two-parameter weight function w(r), where r is the distance measured from the center point of lattice site to which population pressure p is assigned. In our model, this weight function is characterized by either linear or exponential functions using parameters wₘₐₓ=w₁ and wₘᵢₙ=w_{N}_{₁} (for specific formulae, see Appendix 1). FIG. 5C displays the weight function for a specific parameter setting. The value of population pressure is determined according to

$$p = \frac{{\sum }_{k = 1}^{{N}_{1}} {w}_{k} {o}_{k}}{{\sum }_{k = 1}^{{N}_{1}} {w}_{k}} , \qquad ( 2 )$$

where the occupancy rate is computed as oₖ=N_{o,k}/Nₖ, with N_{o,k} being the number of occupied lattice sites, while Nₖ is the overall number of lattice sites inside the k-th layer. In addition to the population pressure p, quadrant pressures pᵢ, i=1, 2, 3, 4; are also computed for each lattice site. Quadrant pressures characterize the occupancy rates inside the four quadrants (see FIG. 5B) of the extended neighborhood corresponding to the four immediate neighbors of the lattice site at which p is computed. Quadrant pressures are determined as follows

$${p}_{i} = \frac{{\sum }_{k = 1}^{{N}_{1}} {w}_{k} {o}_{i , k}}{{\sum }_{k = 1}^{{N}_{1}} {w}_{k}} , \qquad ( 3 )$$

where o_{i,k}=N_{o,i,k}/N_{i,k} is the occupancy rate inside the k-th layer of the i-th quadrant, with N_{o,i,k} being the number of occupied lattice sites inside the k-th layer of the i-th quadrant and N_{i,k} being the total number of lattice sites inside the k-th layer of the i-th quadrant. Note that N_{i,k} is the same for all k, due to symmetry. Lattice sites along the boundary of two quadrants are assigned to both quadrants, since each lattice site is either occupied or empty.

### Division Process

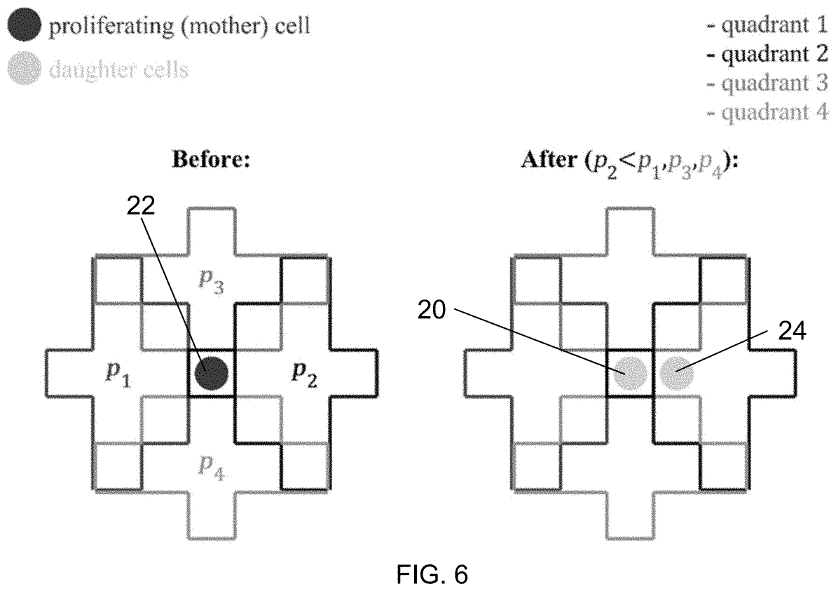

During division, one of the daughter cells is placed into the lattice site occupied by the mother cell, while the other daughter is placed into one of the four immediate neighbor lattice sites. In the following, the immediate neighbor lattice site chosen for the latter daughter is referred to as target site. In contrast to the method for daughter placement in (Sipahi and Zupanc 2018), where the target site was selected randomly from the empty immediate neighbors, here the immediate neighbor with smallest corresponding quadrant pressure is chosen as target site. The process of daughter placement is illustrated in FIG. 6. After division, one of the daughter cells 20 occupies the lattice site of the mother cell 22, while the other daughter cell 24 is placed into the immediate neighbor lattice site with smallest quadrant pressure (i.e., into the target site).

It is important to note that, in contrast with (Sipahi and Zupanc 2018), the herein detailed rules allow mitosis even when all four immediate neighbor lattice sites are occupied, as long as there is at least one empty lattice site in the extended neighborhood. When all immediate neighbor sites are occupied then, in order to realize division, the cell at target site must be shifted away. This shifting is carried out according to the following rules:
a) Inside the quadrant of daughter placement, i.e., the quadrant where the target site is located, the empty lattice site that is the closest to the target site is filled during shifting.
b) One out of all possible shortest routes between the target site and selected empty lattice site is chosen with uniform probability. For details on the computation of these shortest routes, see Appendix 3.
c) Along this chosen route, referred to as shifting route in the following, cells are shifted from the target site toward the selected empty lattice site so that the empty lattice site is filled, and the target site is emptied.

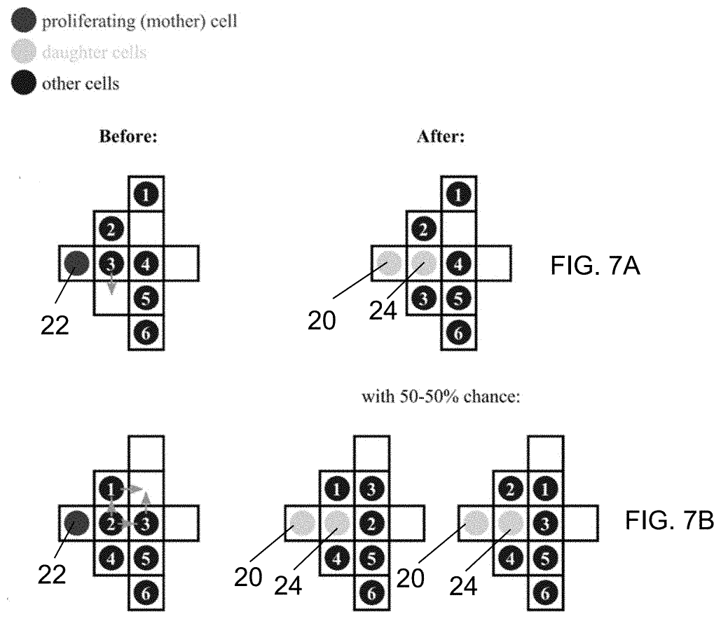

The process of shifting is illustrated in FIGS. 7A and 7B for two different scenarios. A mother cell 22 divides and gives rise to two daughter cells 20, 24. In the FIG. 7A case, before mitosis the target site is occupied by cell No. 3. During shifting, this cell is moved towards the empty space closest to the target site thereby freeing up the target site and filling the closest empty space. Shifting is carried out along the shortest route indicated by green arrow. In the FIG. 7B case, before mitosis the target site is occupied by cell No. 2. Two shortest routes, indicated by the arrows, exist between target site and closest empty space, out of which one is chosen randomly with uniform probability.

Because it is possible that in a time iteration step two or more different cells that undergo mitosis claim the same target site for their daughters, an order must be established for the division of cells that undergo mitosis in the same time iteration step. We arbitrarily defined an order in which daughter placement is followed by shifting. As part of our simulations, we sort, during each time iteration step, all cells that undergo mitosis according to the quadrant pressure corresponding to their target site. We assume that cells with smaller quadrant pressure will undergo mitosis faster, thus we carry out daughter placement and shifting in an ascending order of quadrant pressure. Within a single iteration step, all population pressure and quadrant pressure values are updated after each division (daughter placement and shifting).

### Boundary Conditions

In order to carry out simulations using the above detailed lattice and rules, the model is equipped with boundary conditions. Given that the 2D lattice corresponds to an axisymmetric 3D grid, at the lower boundary (i.e., at y=0) the cellular composition of the lattice is mirrored around the axis x. With this boundary condition (BC), it is taken into account that no cells can be found inside the central canal. Note that due to mirroring around x, the rules detailed under Section 4.1.2 cannot result in daughter placement or shifting across x. Consequently, these rules cannot result in the violation of the mirroring BC. In addition to the mirroring BC around x, the occupancy of left, top and right boundaries must be provided, for each time iteration, outside the lattice, within a margin of N₁ lattice sites. This is necessary because otherwise population pressure values cannot be computed according to Eqs. (2) and (3).

### Initial Conditions

To make the execution of simulations feasible, an initial cellular composition for the entire lattice is provided. This means the precise specification of location and state values of all agents inside the lattice at the initial time iteration t=0. Note that BCs are also provided at t=0.

### Outline of Algorithm

In the following, we provide an outline of the algorithm that was developed according to the above detailed model.

### Initialization

a) Linear or exponential characteristics is selected for functions P_{M}(p), P_{D}(p), and w(r).
b) Values of parameters Nₗ, wₘᵢₙ, wₘₐₓ, R_{c}, P_{A,max}, P_{S,max}, P_{M,min}, P_{M,max}, P_{D,min}, P_{D,max}, N_{q}, N, N_{d} are provided.
c) Lattice is defined: number of lattice sites in the horizontal (axial) direction Lₓ, and number of lattice sites in the vertical (radial) direction L_{y} are provided.
d) Number of time iterations tₘₐₓ is provided.
e) Boundary conditions are provided for all time iterations.
f) Initial conditions (initial lattice composition) are provided.

### Time Iteration

for t=0: t_{end}−1
1) Compute population pressure and quadrant pressure values according to Eq. (2)-(3) for each lattice site on the lattice using data from time iteration t.
2) Compute new state values at t+1 for each agent according to Table 1 and the flowchart in FIG. 2.
3) Apply all outcomes which do not result in division, place the rest of the outcomes in the empty set S.
while |S|>0
a) Sort outcomes in S according to their lowest quadrant pressure in ascending order, apply random ordering for outcomes with equal quadrant pressure.
b) Carry out shifting (if necessary) and daughter placement for the mother cell corresponding to the first element of the sorted S set, if shifting is not possible (but necessary) then cancel division (keep state value corresponding to t).
c) Take out (drop) the first element from set S.
d) Recompute quadrant pressures for all remaining outcomes in set S.

end

end

### Simulation

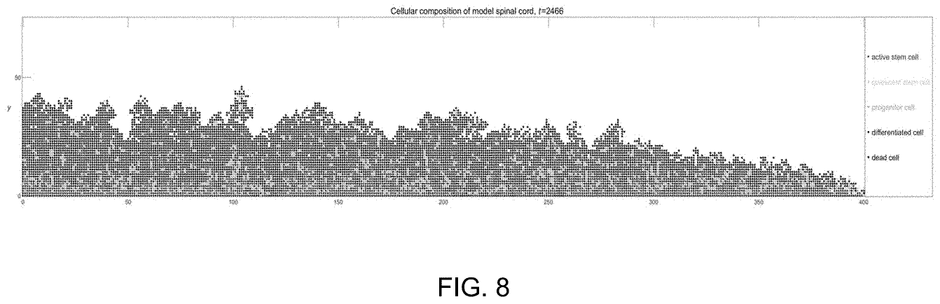

To demonstrate what results our invention can produce, one exemplary simulation outcome at a particular, final time iteration is shown in FIG. 8. This simulation was initialized as follows.
a) Linear characteristics was assumed for functions P_{M}(p), P_{D}(p), and w(r).
b) Parameter values were chosen as N₁=4, wₘᵢₙ=0.05, wₘₐₓ=¹, R_{c}=1, P_{A,max}=0.8, P_{S,max}=1, P_{M,min}=0, P_{M,max}=1, P_{D,min}=0.25, P_{D,max}=0.75, N_{q}=3, N=5, N_{d}=10.
c) Lattice dimensions were specified as Lₓ=401, Lₓ=76.
d) Number of time iterations were determined as t_{end}=2466.
e) Boundary conditions were set according to the following: left boundary margin was completely occupied; top and right boundary margins were completely empty throughout the course of simulation.
f) Initial conditions were chosen as follows: at time iteration t=0 the entire lattice was empty, except for the lattice site in the left bottom corner of the lattice, at position (x, y)=(0,0), where a quiescent stem cell with q₀=1 was placed.

The simulation results in FIG. 8 show the cellular composition of the spinal cord for a single angular slice along the central canal. Different colors indicate different cell types in the figure, blank spaces reflect (empty) intercellular space. The depicted result is in line with biological measurements produced in the spinal cord of adult brown ghost knifefish (Apteronotus leptorhynchus). In particular, as detailed in (Sirbulescu et al. 2017), a thin layer, called ependymal layer, along the central canal (close to y=0) consists almost entirely of stem cells. More peripheral areas (the so-called parenchyma) are characterized by a significantly lower concentration of stem and progenitor cells, and an abundance of differentiated cells, as found in biological tissue. Furthermore, close to the tip of the spinal cord (close to x=400), the stem cell versus differentiated cell ratio is higher in vertical sections (columns of lattice sites) than in those sections that are located further from the tip.

### Advantages

Tissue growth is based on the behavior of a finite number of discrete cells and the interactions among them. These dynamics are determined by molecular and cellular mechanisms, as well as by stochastic processes, which can be translated into a set of rules and associated probabilities. Thus, for modeling of these growth dynamics, CA provides a framework superior to the traditionally used differential-equation approach because its agents, like biological cells, are finite and distinct, and their local interactions depend on sets of rules. By contrast, in approaches based on differential equations space, time, and state are all continuous. The differential equation framework can be particularly prohibitive to the incorporation of rule-based interactions, as they often lead to jump conditions or non-smoothness in differential equations, resulting in systems that are difficult to simulate. Another major advantage of CA for modeling of tissue growth is that the specific rules and the associated probabilities can be readily modified to adjust for differences in the type or developmental stage of tissue. This flexibility makes the designed model a versatile tool for applications in drug discovery and tissue engineering.

### REFERENCES CITED

Sirbulescu R. F., Ilie [symbol] I., Meyer A., Zupanc G. K. H., 2017. Additive neurogenesis supported by multiple stem cell populations mediates adult spinal cord development: A spatiotemporal statistical mapping analysis in a teleost model of indeterminate growth. Dev. Neurobiol. 77, 1269-1307.
Ilie [symbol] I., Sipahi R., Zupanc G. K. H., 2018. Growth of adult spinal cord in knifefish: Development and parametrization of a distributed model. J. Theor. Biol. 437, 101-114.
Sipahi R., Zupanc G. K. H., 2018. Stochastic cellular automata model of neurosphere growth: roles of proliferative potential, contact inhibition, cell death, and phagocytosis. J. Theor. Biol. 445, 151-165.

### Computer Implementation

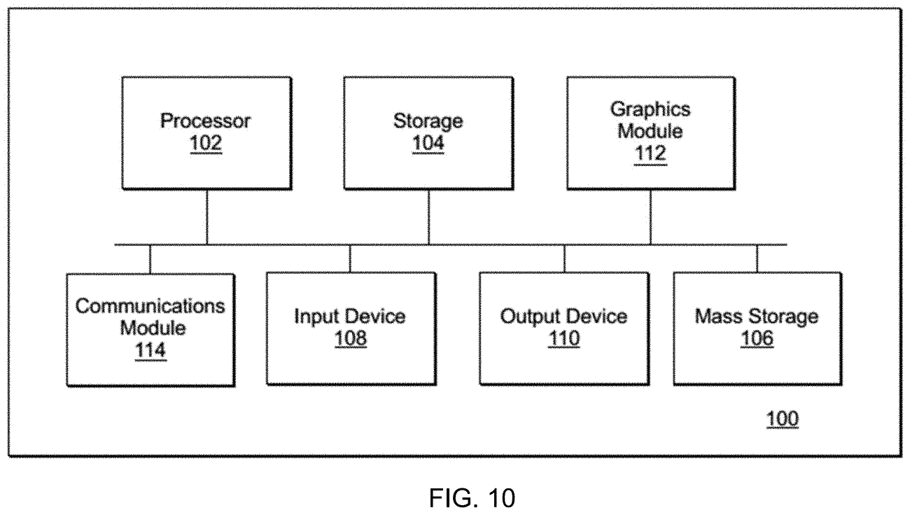

The methods, operations, modules, and systems described herein may be implemented in one or more computer programs executing on a programmable computer system. FIG. 10 is a simplified block diagram illustrating an exemplary computer system 100, on which the computer programs may operate as a set of computer instructions. The computer system 100 includes at least one computer processor 102, system memory 104 (including a random access memory and a read-only memory) readable by the processor 102. The computer system 100 also includes a mass storage device 106 (e.g., a hard disk drive, a solid-state storage device, an optical disk device, etc.). The computer processor 102 is capable of processing instructions stored in the system memory or mass storage device. The computer system additionally includes input/output devices 108, 110 (e.g., a display, keyboard, pointer device, etc.), a graphics module 112 for generating graphical objects, and a communication module or network interface 114, which manages communication with other devices via telecommunications and other networks.

Each computer program can be a set of instructions or program code in a code module resident in the random access memory 104 of the computer system 100. Until required by the computer system 100, the set of instructions may be stored in the mass storage device 106 or on another computer system and downloaded via the Internet or other network.

Having thus described several illustrative embodiments, it is to be appreciated that various alterations, modifications, and improvements will readily occur to those skilled in the art. Such alterations, modifications, and improvements are intended to form a part of this disclosure, and are intended to be within the spirit and scope of this disclosure. While some examples presented herein involve specific combinations of functions or structural elements, it should be understood that those functions and elements may be combined in other ways according to the present disclosure to accomplish the same or different objectives. In particular, acts, elements, and features discussed in connection with one embodiment are not intended to be excluded from similar or other roles in other embodiments.

Additionally, elements and components described herein may be further divided into additional components or joined together to form fewer components for performing the same functions. For example, the computer system 100 may comprise one or more physical machines, or virtual machines running on one or more physical machines. In addition, the computer system 100 may comprise a cluster of computers or numerous distributed computers that are connected by the Internet or another network.

Accordingly, the foregoing description and attached drawings are by way of example only, and are not intended to be limiting.

Appendix 1
Two-parameter functions used for probabilities and weights

| | | Probability of daughter cell | Weight function of |
|---|---|---|---|
| | Probability of mitosis | death | extended neighborhood |
| Exp. | ${P}_{M} ( p ) = {{P}_{M , max} ( \frac{{P}_{M , min}}{{P}_{M , max}} )}^{p}$ | ${P}_{D} ( p ) = {{P}_{D , min} ( \frac{{P}_{D , max}}{{P}_{D , min}} )}^{p}$ | $w ( r ) = {( \frac{{w}_{max}^{{N}_{l} - r}}{{w}_{min}^{1 - r}} )}^{\frac{1}{{N}_{l} - 1}}$ |
| Lin. | P_{M}(p) = P_{M,max}(1 − p) + P_{M,min} p | P_{D}(p) = P_{D,min}(1 − p) + P_{D,max} p | $w ( r ) = {w}_{max} ( 1 - \frac{r - 1}{{N}_{l} - 1} ) + {w}_{min} \frac{r - 1}{{N}_{l} - 1}$ |

### Appendix 2: Derivation of Hyperbolic Spatial Dependence of Stem-Cell-Related Probabilities

Since the diffusive factor is evenly distributed inside the spinal cord, its overall value in a particular lattice site in the three-dimensional grid is proportional to the volume of the lattice site which can be computed as

V(R)=A(R)Δx, (A 2.1)

where Δx is the size of square lattice sites in the associated two-dimensional grid and

$$A ( R ) = \frac{2 πΔ x}{{N}_{θ}} R \qquad ( A 2.2 )$$

is the area of the transversal section of lattice sites, whose center points are located with R distance from the center line of central canal. The number of angular segments in the three-dimensional model is denoted by N_{θ}. Due to that in each lattice site the probability of activation and symmetric division of stem cell are inversely proportional to the overall value of diffusive factor inside the lattice site's volume

$${P}_{χ} ( R ) = \frac{c}{V ( R )} , \qquad ( A 2.3 )$$

with χ=A, S and c being a scaling factor. In order to parameterize these functions by their maximum values along the central canal, we require

P_{χ}(R_{c}+Δy/2)=P_{χ,max}, (A 2.4)

where Δy=Δx, due to the square grid. From Eq. (A 2.4) c can be expressed and plugged into Eq. (A 2.3) which becomes

$${P}_{χ} ( R ) = \frac{{P}_{χ , max}}{2 R \text{/} ( 2 {R}_{c} + Δ x )} . \qquad ( A 2.5 )$$

Due to that R=R_{c}+y+Δy/2, furthermore that Δx=1 is employed in our cellular automata model, one ends up with the formula in Eq. (1).

### Appendix 3: Computation of Shifting Routes

A shifting route is one shortest route chosen randomly with uniform probability from all possible shortest routes between the target site and the empty lattice site closest to it within the quadrant of daughter placement. It is assumed that routes between the center points of these two lattice sites consist of only horizontal and vertical steps, with each step size being equal to the size of square lattice sites. Consequently, the horizontal steps within shortest routes must sum to the horizontal distance dₓ, between the two endpoints of these routes. The same holds true for vertical steps where vertical distance of the two endpoints of all possible routes is denoted by d_{y}. Since in our model square lattice sites have unit size (Δx=Δy=1), distances dₓ and d_{y} are equal to the overall number of horizontal and vertical steps in shortest routes, respectively. In order to find all possible shortest routes, one has to solve a combinatorial partitioning problem, where a total of dₓ+d_{y} number of steps have to be made consisting of exactly dₓ number of horizontal and d_{y} number of vertical steps. As a result, the overall number of different shortest routes, i.e., different sequences of horizontal and vertical steps, can be computed as

$${N}_{sr} = \frac{( {d}_{x} + {d}_{y} ) !}{{d}_{x} ! {d}_{y} !} . \qquad ( A 3.1 )$$

1. A computer-implemented method of predicting stem-cell-driven growth of biological spinal cord tissue under given conditions, comprising:
(a) electronically accessing a computational model for simulating stem-cell driven spinal cord tissue growth, said model based on a cellular automata (CA) framework comprising a plurality of lattice sites, wherein each lattice site is empty or contains a cell having one of four state values: stem cell, progenitor cell, differentiated cell, or dead cell; said model including a plurality of interaction rules for predicting the state value of each lattice site at a next time iteration based on the state values of cells in neighboring lattice sites at a current time iteration;
(b) electronically receiving input data specifying an initial lattice composition and lattice boundary conditions;
(c) electronically running a simulation on the computational model based on the input data for a plurality of time iterations to predict development of the biological spinal cord tissue over the time iterations; and
(d) electronically outputting data on the development of the biological spinal cord tissue.

2. The method of claim 1, further comprising predicting the effect of experimental or pharmacological interventions to improve spinal cord regrowth after injury using the data output in (d).

3. The method of claim 1, wherein the CA framework comprises a two-dimensional (2D) lattice of square lattice sites derived from a three-dimensional (3D) cylindrical model.

4. The method of claim 1, wherein the interaction rules include rules for cell activation, division, differentiation, apoptosis, and phagocytosis.

5. The method of claim 1, wherein the model uses population pressure p as a parameter for analyzing each lattice site, said population pressure being based on a cellular density of an extended neighborhood of lattice sites beyond a quadrant of immediate neighbors of each lattice site.

6. The method of claim 1, wherein the model utilizes probability of death of daughter cell P_{D}(p) and probability of mitosis P_{M}(p) functions to characterize apoptosis and mitosis of cells, respectively.

7. The method of claim 1, wherein the probability of stem cell activation P_{A}(y) in the model is dependent on a radial distance y from a central canal surface of the CA framework.

8. The method of claim 1, wherein stem cells undergo symmetric division in the model with probability P_{S}(y), which depends on a radial distance y from a central canal surface of the CA framework.

9. A computer system, comprising:
at least one processor;
memory associated with the at least one processor; and
a program supported in the memory for predicting stem-cell-driven growth of biological spinal cord tissue under given conditions, the program containing a plurality of instructions which, when executed by the at least one processor, cause the at least one processor to:
(a) electronically access a computational model for simulating stem-cell driven spinal cord tissue growth, said model based on a cellular automata (CA) framework comprising a plurality of lattice sites, wherein each lattice site is empty or contains a cell having one of four state values: stem cell, progenitor cell, differentiated cell, or dead cell; said model including a plurality of interaction rules for predicting the state value of each lattice site at a next time iteration based on the state values of cells in neighboring lattice sites at a current time iteration;
(b) electronically receive input data specifying an initial lattice composition and lattice boundary conditions;
(c) electronically run a simulation on the computational model based on the input data for a plurality of time iterations to predict development of the biological spinal cord tissue over the time iterations; and
(d) electronically output data on the development of the biological spinal cord tissue.

10. The computer system of claim 9, wherein the data output in (d) is used for predicting the effect of experimental or pharmacological interventions to improve spinal cord regrowth after injury.

11. The computer system of claim 9, wherein the CA framework comprises a two-dimensional (2D) lattice of square lattice sites derived from a three-dimensional (3D) cylindrical model.

12. The computer system of claim 9, wherein the interaction rules include rules for cell activation, division, differentiation, apoptosis, and phagocytosis.

13. The computer system of claim 9, wherein the model uses population pressure p as a parameter for analyzing each lattice site, said population pressure being based on a cellular density of an extended neighborhood of lattice sites beyond a quadrant of immediate neighbors of each lattice site.

14. The computer system of claim 9, wherein the model utilizes probability of death of daughter cell P_{D}(p) and probability of mitosis P_{M}(p) functions to characterize apoptosis and mitosis of cells, respectively.

15. The computer system of claim 9, wherein the probability of stem cell activation P_{A}(y) in the model is dependent on a radial distance y from a central canal surface of the CA framework.

16. The computer system of claim 9, wherein stem cells undergo symmetric division in the model with probability P_{S}(y), which depends on a radial distance y from a central canal surface of the CA framework.

17. A computer program product residing on a non-transitory computer readable medium having a plurality of instructions stored thereon which, when executed by a computer processor, cause that computer processor to: (a) electronically access a computational model for simulating stem-cell driven spinal cord tissue growth, said model based on a cellular automata (CA) framework comprising a plurality of lattice sites, wherein each lattice site is empty or contains a cell having one of four state values: stem cell, progenitor cell, differentiated cell, or dead cell; said model including a plurality of interaction rules for predicting the state value of each lattice site at a next time iteration based on the state values of cells in neighboring lattice sites at a current time iteration; (b) electronically receive input data specifying an initial lattice composition and lattice boundary conditions; (c) electronically run a simulation on the computational model based on the input data for a plurality of time iterations to predict development of the biological spinal cord tissue over the time iterations; and (d) electronically output data on the development of the biological spinal cord tissue.

18. The computer program product of claim 17, wherein the data output in (d) is used for predicting the effect of experimental or pharmacological interventions to improve spinal cord regrowth after injury.
